(19) United States
(12) Patent Application Publication
(10) Pub. No.: US 2020/0096496 A1
(43) Pub. Date: Mar. 26, 2020

(54) IODIDE DETECTION IN BLOOD PLASMA SAMPLES

(71) Applicant: Faraday Pharmaceuticals, Inc., Sealttle, WA (US)

(72) Inventors: Emily VANDENEKART, Kirkland, WA (US); Mark B. Roth, Seattle, WA (US)

(21) Appl. No.: 16/621,116

(22) PCT Filed: Jun. 14, 2018
(86) PCT No.: PCT/US18/37636

§ 371 Date: Dec. 10, 2019

Related U.S. Application Data
(60) Provisional application No. 62/519,540, filed on Jun. 14, 2017.

(51) Int. Cl. G01N 33/49; G01N 1/34; G01N 30/96

(52) U.S. Cl. CPC G01N 33/49; G01N 30/96; G01N 1/34

(57) ABSTRACT

The present invention relates to methods for measuring a concentration of iodide in a plasma sample obtained from a subject by removing an amount of protein from the plasma sample, e.g. by precipitating protein from the plasma sample, and then measuring the concentration of the iodide in the plasma sample with ion chromatography.

### CROSS REFERENCE TO RELATED APPLICATIONS

This application claims priority to U.S. Provisional Application No. 62/519,540, filed on Jun. 14, 2017, which is hereby incorporated by reference in its entirety.

### FIELD OF THE INVENTION

This invention relates to methods for measuring a concentration of iodide in a plasma sample obtained from a subject using, e.g., ion chromatography.

### BACKGROUND OF THE INVENTION

Since its discovery in 1811, iodine has been intensively studied. It is thought that the very first human trial on a defined chemical showed in 1826 that iodine could be used to prevent and treat goiter. Since then, a large body of research has led to the understanding that iodine in the form of iodide is required for thyroid hormone synthesis. Iodine's main role in animal biology is as constituents of the thyroid hormones, thyroxine (T4) and triiodothyronine (T3). These are made from addition condensation products of the amino acid tyrosine, and are stored prior to release in an iodine-containing protein called thyroglobulin. T4 and T3 contain four and three atoms of iodine per molecule, respectively. The thyroid gland actively absorbs iodine from the blood to make and release these hormones into the blood, actions regulated by a second hormone TSH from the pituitary.

Iodide refers to the ion form of iodine. Because iodide contributes to critical biological functions in healthy individuals, there exists a need to measure circulating iodide concentrations with a high degree of accuracy and sensitivity. The present invention meets this need by providing a novel method of measuring iodide concentrations in plasma samples with a high degree of sensitivity and accuracy.

### BRIEF SUMMARY OF THE INVENTION

The present invention provides inter alia methods for measuring a concentration of iodide in a plasma sample obtained from a subject by removing an amount of protein from the plasma sample and then measuring the concentration of the iodide in the plasma sample with ion chromatography.

Particular methods of the present invention are directed to a method for measuring a concentration of iodide in a plasma sample obtained from a subject, comprising the steps of: (a) removing an amount of protein from the plasma sample; and (b) measuring the concentration of the iodide in the sample with ion chromatography. In some embodiments, the subject is a mammal. In particular embodiments, the plasma sample is obtained from the subject following administration of iodide to the subject.

In certain embodiments, the plasma sample is obtained from the subject prior to the administration of iodide to the subject. In some embodiments, the subject is diagnosed with or at risk of hypoxia, ischemia or reperfusion injury. In particular embodiments, the subject is diagnosed with or at risk of stroke, heart attack, or blood loss. In certain embodiments, the subject is undergoing or is scheduled to undergo cell, tissue, or organ transplantation. the ion chromatography comprises conductivity detection.

In particular embodiments, the ion chromatography comprises UV/VIS detection. In certain embodiments, the ion chromatography comprises amperometry. In some embodiments, the ion chromatography can detect a concentration of the iodide below 5 parts per million in a plasma sample. In certain embodiments, the ion chromatography can detect a concentration of the iodide that is between about 0.001 and about 500 parts per billion. In particular embodiments, the ion chromatography can detect a concentration of the iodide that is between about 2.5 and about 200 parts per billion. In some embodiments, the plasma sample comprises a volume that is between about 0.05 ml and about 2 ml. In certain embodiments, the plasma sample comprises a volume that is between 0.1 ml and 1 ml. In particular embodiments, the volume is about 0.5 ml.

In some embodiments, the step of (a) removing an amount of protein from the plasma sample is performed by precipitating the amount of protein from the sample. In particular embodiments, precipitating the amount of protein from the sample comprises protein precipitation by an organic solvent, protein precipitation by a metal ion, isoelectric point precipitation, protein precipitation with miscible solvents, precipitation with non-ionic hydrophilic polymers, or flocculation by polyelectrolytes. In some embodiments, precipitating the amount of protein from the sample comprises the technique of protein precipitation by organic solvent.

In particular embodiments, the step of (a) removing an amount of protein from the plasma sample comprises the steps of: (i) contacting the plasma sample with a protein precipitation agent; (ii) centrifuging the plasma sample in a manner sufficient to generate a pellet and a supernatant in the plasma sample, wherein the pellet comprises the amount of protein, and wherein the supernatant comprises the remainder of the plasma sample; and (iii) collecting the supernatant and discarding the pellet; thereby removing the amount of protein from the plasma sample. In some embodiments, the plasma sample is mixed with the protein precipitation agent prior to step (ii). In certain embodiments, the protein precipitation agent comprises pure acetonitrile, acetone, trichloroacetic acid (TCA), or phenol/chloroform. In particular embodiments, the protein precipitation agent comprises pure acetonitrile.

In particular embodiments, the amount of protein removed from the sample is at least 50% of the protein present in the sample prior to the step of (a) removing an amount of protein from the plasma sample. In some embodiments, the amount of protein is at least 80% of protein present in the sample prior to step (a). In certain embodiments, the amount of protein is at least 90% of protein present in the sample prior to step (a).

In certain embodiments, the step of (a), removing an amount of protein from the plasma sample, comprises the steps of: (i) contacting the plasma sample with pure acetonitrile and mixing the pure acetonitrile with the plasma sample; (ii) centrifuging the plasma sample in a manner sufficient to generate a pellet and a supernatant in the plasma sample, wherein the pellet comprises the amount of protein, and wherein the supernatant comprises the remainder of the plasma sample; and (iii) collecting the supernatant and discarding the pellet; thereby removing an amount of protein from the plasma sample; and wherein the step of (b), measuring the concentration of the iodide in the sample with ion chromatography, comprises measuring the amount of iodide in the supernatant using an amperometric detector; wherein the volume of the plasma sample is about 0.5 ml.

Particular embodiments of the present invention are drawn to a method for determining a concentration of iodide in the blood plasma of a subject at two or more time points over a period of time comprising the steps of: (I) administering an amount of iodide to the subject; (II) collecting two or more plasma samples from the subject at the two or more time points within the period of time; (III) measuring the concentration of the iodide in the two or more plasma samples by measuring the concentrations of iodide on each plasma sample by any of the methods described herein for the two or more plasma samples. In some embodiments, the period of time is between about 1 minute and about two weeks. In particular embodiments, the subject is continuously administered iodide intravenously over the period of time. In some embodiments, the subject is administered iodide prior to the period of time. In particular embodiments, the period of time starts from the time the iodide is administered to the subject. In certain embodiments, the iodide is administered to the subject orally, nasally, intravenously, intramuscularly, ocularly, transdermally, or subcutaneously. In particular embodiments, the iodide is administered intravenously. In some embodiments, the iodide is administered orally.

Certain embodiments of the present invention are drawn to a method for determining if an amount of iodide administered to a subject should be increased or decreased by measuring a concentration of iodide in the blood plasma of a subject over a period of time comprising the steps of: (I) administering the amount of iodide to the subject; (II) collecting a plasma sample from the subject; (III) measuring the concentration of the iodide in the plasma sample by performing a method described herein, thereby obtaining a measurement of the concentration; and (IV) comparing the measurement of the concentration to a predetermined preferred range of plasma iodide concentrations; wherein if the measurement of the concentration falls within the predetermined preferred range, then the amount should not be increased or decreased; wherein if the measurement of the concentration falls below the predetermined preferred range, then the amount of iodide should be increased, and wherein if the measurement of the concentration falls above the predetermined preferred range, then the amount of iodide should be decreased. In particular embodiments, the plasma sample is taken from the subject 30 minutes after the iodide is administered. In some embodiments, the predetermined preferred range is 50-100 ppb of iodide.

In embodiments of any of the methods disclosed herein, iodide is extracted from plasma obtained from a subject and assayed against a spiked matrix calibration curve or standard curve using IC-ECD.

### BRIEF DESCRIPTION OF THE DRAWINGS

FIG. 1 shows the general set-up a flow path for the Metrohm 850 Professional IC AnCat (top) and the involved components (bottom). Adapted from Metrohm 850 Professional IC AnCat manual.

FIG. 2 shows the general electric path for the three electrode configuration used with the 850 IC Amperometric Detector Compact.

FIGS. 3A-3C show standard iodide curves. FIG. 3A shows the full trace results from the 20 minute run. FIG. 3B shows a closer view of the standard curve peaks. FIG. 3C shows a plot of concentration (x-axis) by amplitude (y-axis), RSD: 1.21% CoEff: 0.99991.

FIG. 4A and FIG. 4B show the results of iodide detection in human plasma. FIG. 4A shows the full trace from the 20 minute run. FIG. 4B shows a close up of the sample peaks. Curves in FIG. 4B represent the diluent blank, baseline plasma, 50 parts per billions (ppb) plasma, and 100 ppb plasma, from the bottom curve to the top curve, respectively.

FIGS. 5A-5C show standard iodide curves. FIG. 5A shows the full trace results from the 20 minute run. FIG. 5B shows a closer view of the standard curve peaks. FIG. 5C shows a plot of concentration (x-axis) by amplitude (y-axis).

### DETAILED DESCRIPTION OF THE INVENTION

The present invention is based, in part, on the surprising and unexpected finding that concentrations of iodide can be accurately measured with a high degree of sensitivity by removing protein from blood plasma prior to measuring the iodide concentration by ion chromatography, e.g. amperometric ion chromatography, and furthermore, that the protein can be removed from the plasma without altering the concentration of the iodide. The present invention further provides uses and applications of the described methods.

### Definitions and Abbreviations

Unless otherwise defined herein, scientific and technical terms used in this application shall have the meanings that are commonly understood by those of ordinary skill in the art. Generally, nomenclature used in connection with, and techniques of, chemistry, molecular biology, cell and cancer biology, immunology, microbiology, pharmacology, and protein and nucleic acid chemistry, described herein, are those well-known and commonly used in the art.

As used herein, the following terms have the meanings ascribed to them unless specified otherwise.

The term “including” is used to mean “including but not limited to.” “Including” and “including but not limited to” are used interchangeably.

The words “a” and “an” denote one or more, unless specifically noted.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length. In any embodiment discussed in the context of a numerical value used in conjunction with the term “about,” it is specifically contemplated that the term about can be omitted.

Unless the context requires otherwise, throughout the present specification and claims, the word “comprise” and variations thereof, such as, “comprises” and “comprising” are to be construed in an open, inclusive sense, that is as “including, but not limited to”.

By “consisting of” is meant including, and limited to, whatever follows the phrase “consisting of” Thus, the phrase “consisting of” indicates that the listed elements are required or mandatory, and that no other elements may be present.

By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment” or “an embodiment” means that a particular feature, structure or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

An “increased” or “enhanced” amount is typically a “statistically significant” amount, and may include an increase that is 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20, 30, 40, or 50 or more times (e.g., 100, 500, 1000 times) (including all integers and decimal points in between and above 1, e.g., 2.1, 2.2, 2.3, 2.4, etc.) an amount or level described herein.

A “decreased” or “reduced” or “lesser” amount is typically a “statistically significant” amount, and may include a decrease that is about 10%, 20%, 30%, 40%, 50%, 60%, 70%, 80%, 85%, 90%, 95%, 99%, or 100% (including all integers and decimal points in between) less than an amount or level described herein.

A “composition” can comprise an active agent, e.g., an iodide compound, and a carrier, inert or active, e.g., a pharmaceutically acceptable carrier, diluent or excipient. A composition may be a pharmaceutical composition. In particular embodiments, the compositions are sterile, substantially free of endotoxins or non-toxic to recipients at the dosage or concentration employed.

“Pharmaceutically acceptable carrier, diluent or excipient” includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent or emulsifier which has been approved by the United States Food and Drug Administration as being acceptable for use in humans or domestic animals.

The term “biological matter” refers to any living biological material, including cells, tissues, organs, and/or organisms, and any combination thereof. It is contemplated that the methods of the present invention may be practiced on a part of an organism (such as in cells, in tissue, and/or in one or more organs), whether that part remains within the organism or is removed from the organism, or on the whole organism. Moreover, it is contemplated in the context of cells and tissues that homogenous and heterogeneous cell populations may be the subject of embodiments of the invention. The term “in vivo biological matter” refers to biological matter that is in vivo, i.e., still within or attached to an organism. Moreover, the term “biological matter” will be understood as synonymous with the term “biological material.” In certain embodiments, it is contemplated that one or more cells, tissues, or organs is separate from an organism. The term “isolated” can be used to describe such biological matter. It is contemplated that the methods of the present invention may be practiced on in vivo and/or isolated biological matter.

The terms “mammal” and “subject” includes human and non-human mammals, such as, e.g., a human, mouse, rat, rabbit, monkey, cow, hog, sheep, horse, dog, and cat.

The terms “tissue” and “organ” are used according to their ordinary and plain meanings. Though tissue is composed of cells, it will be understood that the term “tissue” refers to an aggregate of similar cells forming a definite kind of structural material. Moreover, an organ is a particular type of tissue. In certain embodiments, the tissue or organ is “isolated,” meaning that it is not located within an organism.

As used in the specification and appended claims, unless specified to the contrary, the following terms have the meaning indicated:

“Mammal” includes humans and both domestic animals such as laboratory animals and household pets, (e.g., cats, dogs, swine, cattle, sheep, goats, horses, and rabbits), and non-domestic animals such as wildlife and the like.

“Optional” or “optionally” means that the subsequently described event of circumstances may or may not occur, and that the description includes instances where said event or circumstance occurs and instances in which it does not.

“Iodide” and “a reduced form of iodide” both refer to iodide, which has a −1 valence state. In particular embodiments, iodide is present in a salt containing iodide in the −1 oxidation state, e.g., NaI. “A reduced form of iodine” includes iodide. As used herein, “administering iodide” and “administering a composition comprising iodide” are used interchangeably.

“Protein precipitation agent” refers to any agent that is added to a sample, e.g. a plasma sample, to facilitate the precipitation of proteins in the sample. Protein precipitation agents include, but are not limited to, organic solvent, an inorganic salt, a metal salt, an acid, a non-ionic hydrophilic polymer, or a polyelectrolyte.

“Treating” or “treatment” as used herein covers the treatment of the disease, injury, or condition of interest, e.g., tissue injury, in a biological material, e.g., mammal, preferably a human, having the disease or condition of interest, and includes: (i) preventing or inhibiting the disease, injury, or condition from occurring in a biological material, e.g., mammal, in particular, when such mammal is predisposed to the condition but has not yet been diagnosed as having it; (ii) inhibiting the disease, injury, or condition, i.e., arresting its development; (iii) relieving the disease, injury, or condition, i.e., causing regression of the disease or condition; or (iv) relieving the symptoms resulting from the disease, injury, or condition. As used herein, the terms “disease,” “disorder,” and “condition” may be used interchangeably. As used herein, the term “injury” includes unintentional injuries and intentional injuries, including injuries that occur, “at the hand of man,” including injuries associated with medical procedures, such as surgeries and transplantations.

“Blood plasma” and “plasma” as used herein refer to the liquid component of blood that normally holds the blood cells in whole blood in suspension. As used herein, the terms “Blood plasma” and “plasma” are used interchangeably unless otherwise indicated.

Determining Iodide Concentration in Plasma Samples.

Particular embodiments of the present invention are drawn to a method for measuring a concentration of iodide in a blood plasma sample that is obtained from a subject by removing an amount of protein from the plasma sample and then measuring the concentration of the iodide in the sample with ion chromatography. Particular embodiments contemplate that the protein may be removed by a protein purification technique known in the art, provided that that the technique removes a suitable amount of proteins from the plasma sample and provided that the technique does not significantly or substantially alter the iodide concentrations in the plasma. In certain embodiments, the initial plasma sample volume is between 100 and 1000 μL, e.g., between 300 and 700 μL, such as, e.g., 300 μL, 400 or 500 μL. In certain embodiments, the protein is removed from the blood plasma by protein precipitation, optionally by contacting the plasma sample with pure acetonitrile and gently mixing the pure acetonitrile with the plasma sample; centrifuging the plasma sample; and then collecting the resultant supernatant and discarding the pellet, which contains an amount of protein. The resulting supernatant makes up the plasma sample that can be measured with an ion-chromatography technique. Some embodiments contemplate that ion-chromatography techniques capable of measuring iodide concentrations in a sample with a high degree of accuracy are known in the art, and include, for example but not limited to conductivity detection, UV/VIS detection, and amperometric detection, and that one of skill will be able to recognize ion chromatography techniques and systems suitable for the methods of the present invention. In some embodiments, the iodide concentrations of the plasma sample are measured by amperometric ion chromatography.

Some embodiments contemplate that the methods of the present invention are suitable to determine iodide concentrations in plasma from subjects who will be, may be, or have been administered iodide. For example, in certain embodiments, the methods of the present invention are used to measure the concentrations of iodide in a subject over a course of time. This can be achieved, for example, by collecting multiple samples of blood plasma at planned and/or regular time intervals, from a subject who has been, is being, or will be administered iodide. In particular embodiments, the methods of the present invention are used to determine the concentration of iodide in the plasma of a subject that has been administered a therapeutic amount of iodide to determine if the iodide concentration in the subject's plasma falls within a desirable range, and if not, to determine if subsequent doses of iodide should be increased or decreased.

Plasma Samples

Blood plasma is the liquid component of blood that normally holds the blood cells in whole blood in suspension. Plasma makes up about 55% of the body's total blood volume. It is mostly water (up to about 95% by volume), and contains proteins, (e.g. serum albumins, globulins, and fibrinogen), glucose, clotting factors, electrolytes (e.g. Nat, Ca²⁺, Mg²⁺, HCO³⁻, Cl⁻, etc.), hormones, and carbon dioxide. Blood plasma has a density of approximately 1025 kg/m3, or 1.025 g/ml. Plasma also serves as the protein reserve of the human body. It plays a vital role in an intravascular osmotic effect that keeps electrolytes in balanced form and protects the body from infection and other blood disorders.

In particular embodiments, the present invention relates to methods of determining the concentration of iodide in a plasma sample. In some embodiments, the plasma may be obtained from a blood sample taken from a subject by any means known in the art. For example, in some embodiments, the blood plasma is prepared by placing a fresh blood sample from a subject into a tube containing an anticoagulant, and spinning the tube in a centrifuge until the blood cells fall to the bottom of the tube. The blood plasma is then poured or drawn off to collect the plasma sample.

In some embodiments, a plasma sample is obtained from a blood sample. In particular embodiments, the plasma sample comprises a volume of about 10 μl, about 50 μl, about 100 μl, about 200 μl, about 300 μl, about 400 μl, about 500 μl, about 600 μl, about 700 μl, about 800 μl, about 900 μl, about 1 ml, about 1.2 ml, about 1.4 ml, about 1.6 ml, about 1.8 ml, or about 2 ml. In some embodiments, the plasma sample comprises a volume of between about 0.01 ml and about 2 ml, or between about 0.1 ml and about 1 ml. In certain embodiments, the plasma sample comprises a volume of about 500 μl.

Removing Protein From a Plasma Sample

Particular embodiments of the present invention are drawn to methods of measuring iodide concentrations in plasma samples by removing protein from the plasma sample prior to measuring the iodide concentration in the plasma sample with ion chromatography. Certain embodiments contemplate that the protein may be removed by any technique for protein removal from a biological sample that is known in the art, provided that that the technique removes a sufficient amount of proteins from the plasma sample and provided that the technique does not alter the iodide concentrations in the plasma. In some embodiments, the technique removes at least about 50%, at least about 55%, at least about 60%, at least about 65%, at least about 70%, at least about 75%, at least about 80%, at least about 85%, at least about 90%, at least about 95%, at least about 97%, at least about 98%, at least about 99%, or at least about 99.9% of the protein present in the plasma sample.

In certain embodiments, the protein is removed from the blood plasma by a protein precipitation technique prior to determining the iodide concentration by ion chromatography. Some embodiments contemplate that protein precipitation separates proteins by the conversion of soluble proteins to an insoluble state, thus allowing the proteins to be subsequently removed by various means. Particular embodiments contemplate that one of skill in the art will recognize that there are several protein precipitation techniques known in the art that are useful for removing proteins from a plasma sample. One of skill in the art will also recognize that methods of protein precipitation are suitable for use with the methods of iodide detection of the present invention, provided that that the technique removes a sufficient amount of proteins from the plasma sample and provided that the technique does not alter the iodide concentrations in the plasma. Particular embodiments contemplate that these techniques include, but are not limited to, protein precipitation by organic and/or miscible solvents, protein precipitation by “salting out”, protein precipitation by metal ion, isoelectric point precipitation, precipitation with non-ionic hydrophilic polymers, or flocculation by polyelectrolytes.

Particular embodiments contemplate that the different techniques for protein precipitation are defined by the protein precipitation agent added to the solution to induce protein precipitation. In some embodiments, the protein precipitation agent is an organic solvent, an inorganic salt, a metal salt, an acid, a non-ionic hydrophilic polymer, or a polyelectrolyte. In certain embodiments, the protein precipitation agent is pure acetonitrile, acetone, trichloroacetic acid (TCA), or phenol/chloroform. In particular embodiments, the protein precipitation agent is pure acetonitrile.

In some embodiments, protein is removed from the plasma sample by “Salting out” the protein, i.e. performing protein precipitation with inorganic salts. Particular embodiments contemplate that inorganic salts can be added to a solution for the purposes of precipitating the proteins. Salting out is commonly performed with ammonium sulfate. One of skill in the art will recognize that there are potential advantages of ammonium sulfate precipitation. Particular embodiments contemplate that these advantages include (1) at saturation, ammonium sulfate is of sufficiently high molarity to trigger the precipitation of most proteins; (2) ammonium sulfate does not have a large heat of solution, allowing heat generated to be easily dissipated; and (3) the saturated solution of ammonium sulfate (4.04 M at 20° C.) has a density (1.235 g/cm³) that does not interfere with the sedimentation of most precipitated proteins by centrifugation.

In particular embodiments, protein is removed from the plasma sample by protein precipitation with metal ions. One of skill in the art will recognize that metal salts can be used at low concentrations to precipitate proteins and nucleic acids from a solution. Certain embodiments contemplate that this technique is most often performed with polyvalent metal ions, including, but not limited to, Ca²⁺, Mg²⁺, Mn²⁺ or Fe²⁺.

In certain embodiments, protein is removed from the plasma sample by isoelectric precipitation. Precipitation can be achieved by varying the pH of the medium. At low pH's, proteins have a net positive charge because the amide gains an extra proton. At high pH's, they have a net negative charge due to the carboxyl on the protein backbone losing its proton. At their isoelectric point value, a protein has no net charge. This leads to reduced solubility because the protein is unable to interact with the medium and will then fall out of solution. Particular embodiments contemplate that Trichloroacetic Acid (TCA) is commonly used for isoelectric precipitation because it can precipitate proteins at relatively low concentrations (typically around 15% of the solution). The addition of TCA to a solution containing proteins exposes even more of the proteins' hydrophobic structures, resulting in increased precipitation. This technique runs a high risk of leaving denaturing or leaving isolated proteins nonfunctional. Because of this, TCA is generally only used when an active protein isn't needed. In addition, because TCA is an acid, extra steps need to be taken once the precipitation process has been completed, namely adding a base to the solution or washing off the remaining protein with acetone.

In some embodiments, protein is removed from the plasma sample by protein precipitation with miscible and/or organic solvents. Particular embodiments contemplate that the addition of miscible solvents such as ethanol or methanol to a solution may cause proteins in the solution to precipitate. The solvation layer around the protein will decrease as the organic solvent progressively displaces water from the protein surface and binds it in hydration layers around the organic solvent molecules. With smaller hydration layers, the proteins can aggregate by attractive electrostatic and dipole forces. Parameters that may affect the protein precipitation are temperature, pH of the solution, and the protein concentration in solution. Miscible organic solvents decrease the dielectric constant of water, which in effect allows two proteins to come close together.

In some embodiments, protein is removed from the plasma sample by protein precipitation with an organic solvent. Organic solvents include, but are not limited to, acetic acid, acetone, acetonitrile, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme, 1,2-dimethoxy-ethane, dimethyl-formamide, dimethyl sulfoxide, 1,4-dioxane, ethanol, ethyl acetate, ethylene glycol, glycerin, heptane, hexamethylphosphoramide, hexamethylphosphoramide, hexamethylphosphorous triamide, hexane, methanol, methyl t-butyl ether, methylene chloride, N-methyl-2-pyrrolidinone, nitromethane, pentane, petroleum ether, 1-propanol, 2-propanol, pyridine, tetrahydrofuran, toluene, triethyl amine, o-xylene, m-xylene, and p-xylene.

In certain embodiments, protein is removed from the plasma sample by protein precipitation with non-ionic hydrophilic polymers. Certain polymers, such as dextrans and polyethylene glycols, can be used to precipitate proteins. Particular embodiments contemplate that when placed in a solution, these polymers attract water molecules away from the solvation layer around the protein and thus increase the protein-protein interactions to enhance precipitation.

In particular embodiments, protein is removed from the plasma sample by flocculation by polyelectrolytes. Certain embodiments contemplate that alginate, carboxymethycellulose, polyacrylic acid, tannic acid, and polyphosphates can form extended networks between protein molecules in solution, thereby increasing protein-protein interactions and enhancing protein precipitation. The effectiveness of these polyelectrolytes depend on the pH of the solution. Anionic polyelectrolytes are used at pH values less than the isoelectric point. Cationic polyelectrolytes are at pH values above the isoelectric point.

In particular embodiments, a protein precipitation agent contacts the plasma sample, thereby precipitating the protein out of the plasma. In some embodiments, the protein precipitation results in the formation of a pellet the comprises the precipitated protein. When the pellet is formed, the remaining supernatant comprises the remaining plasma, and can be collected for analysis with ion chromatography.

In some embodiments, the protein precipitation agent (e.g., acetonitrile) is added to the plasma sample at a ratio of 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, or 1:10 precipitation agent to plasma sample by volume. In certain embodiments, the protein precipitation agent is at a temperature of about −70° C., about −65° C., about −60° C., about −55° C., about −50° C., about −45° C., about −40° C., about −35° C., about −30° C., about −25° C., about −20° C., about −15° C., about −10° C., about −5° C., about 0° C., about 5° C., about 10° C., about 15° C., about 20° C., about 25° C., or about 30° C. In particular embodiments, the protein precipitation agent is between about −70° C. and about 40° C., between about −20° C. and about 25° C., between about −20° C. and about 0° C., between about 0° C. and about 25° C., between about −20° C. and about 4° C., or between about 4° C. and about 25° C. In some embodiments, the protein precipitation agent is at room temperature.

In certain embodiments, the plasma sample is contacted with a protein precipitation agent, and the mixture is centrifuged. In particular embodiments, the mixture of the plasma sample and the protein precipitation agent is centrifuged at a speed of about 500 rcf, about 1,000 rcf, about 1,500 rcf, about 2,000 rcf, about 2,500 rcf, about 3,000 rcf, about 4,000 rcf, about 5,000 rcf, about 6,000 rcf, about 7,000 rcf, about 8,000 rcf, about 9,000 rcf, about 10,000 rcf, about 11,000 rcf, about 12,000 rcf, about 13,000 rcf, about 14,000 rcf, about 15,000 rcf, about 16,000 rcf, about 17,000 rcf, about 18,000 rcf, about 19,000 rcf, about 20,000 rcf, about 20,817 rcf, about 21,000 rcf, about 25,000 rcf, about 30,000 rcf, about 35,000 rcf, about 40,000 rcf, or about 50,000 rcf, including all integers and ranges in between. In some embodiments, the mixture is centrifuged at a speed of about 1,000 rcf to about 50,000 rcf, about 5,000 rcf to about 25,000 rcf, about 10,000 rcf and 25,000 rcf, or about 15,000 rcf to about 21,000 rcf. In some embodiments, the mixture is spun for 5 seconds, about 15 seconds, about 30 seconds, about 1 minute, about 2 minutes, about 3 minutes, about 4 minutes, about 5 minutes, about 6 minutes, about 7 minutes, about 8 minutes, about 9 minutes, about 10 minutes, about 15 minutes, about 20 minutes, about 30 minutes, about 45 minutes, about 60 minutes, or about 2 hours, including all integers and ranges in between. In a particular embodiment, the mixture of the plasma sample and the protein precipitation agent is spun at about 20,817 rcf for about 2 minutes.

In particular embodiments, the plasma sample is contacted with a protein precipitation agent and the mixture is centrifuged to generate a pellet that comprises an amount of protein and a supernatant that comprises the remainder of the plasma sample, and the supernatant is then gently removed from the pellet. In some embodiments, the supernatant is centrifuged a second time under the same conditions, generating a second pellet and supernatant (i.e. the remainder of the plasma sample), and the supernatant is removed from the pellet.

In some embodiments, the plasma sample is contacted with a protein precipitation agent, and the mixture incubates for a period of time. During the period of time, protein precipitates out of the plasma, thereby generating a pellet comprising an amount of protein. In some embodiments, the period of time is about 5 seconds, about 10 seconds, about 15 seconds, about 30 seconds, about 1 minute, about 2 minutes, about 5 minutes, about 10 minutes, about 15 minutes, about 20 minutes, about 30 minutes, about 45 minutes, about 1 hour, about 2 hours, about 3 hours, about 6 hours, about 12 hours, about 16 hours, about 18 hours, about 24 hours, or about 48 hours, including all ranges and integers in between. In some embodiments, the period of time is between about 5 minutes and about 48 hours, between about 6 hours and 24 hours, or between about 5 minutes and 2 hours. In particular embodiments, the plasma sample is contacted with acetonitrile at room temperature, and protein precipitates out of the plasma sample thereby generating a pellet during a period of time that is between about 1 second and about 5 minutes, about 10 seconds and about 2 minutes, or about 30 seconds and about 1 minute.

In certain embodiments, an amount of protein is removed from the plasma sample prior by protein precipitation, wherein pure acetonitrile is added to the plasma sample at a volumetric ratio of 3:1 acetonitrile to plasma. The plasma and the acetonitrile are mixed, and the mixture is centrifuged for two minutes at 20,817 rcf. The plasma supernatant is removed from the resulting protein pellet. The mixture is spun a second time, and the plasma supernatant is again removed from the resulting pellet. The iodide concentration of the plasma supernatant is then measured with ion chromatography.

Ion Chromatography

Certain embodiments of the present invention are drawn to methods of iodide detection in plasma samples whereby protein is removed from the plasma samples prior to the iodide detection with ion chromatography. Ion chromatography, a form of liquid chromatography, measures concentrations of ionic species by separating them based on their interaction with a resin. Ionic species separate differently depending on species type and size. Sample solutions pass through a pressurized chromatographic column where ions are absorbed by column constituents. As an ion extraction liquid, known as eluent, runs through the column, the absorbed ions begin separating from the column. The retention time of different species determines the ionic concentrations in the sample.

In a typical ion chromatography procedure, a sample of the mixture to be analyzed (analyte) is injected into a carrier fluid (the eluent). The combination is passed through a column containing a stationary fixed material (adsorbent). Compounds contained in the analyte are then partitioned between the stationary adsorbent and the moving eluent/analyte mixture. Different dissolved materials adhere to the adsorbent with different forces. The materials that adhere strongly are moved through the adsorbent more slowly as the eluent flows over them. As the eluent flows through the column the components of the analyte will move down the column at different speeds and therefore separate from one another. A detector is used to analyze the output at the end of the column. There are various detectors, e.g. electrical conductivity detector, UV/VIS detectors, and amperometric detectors. Each time analyte molecules/ions emerge from the chromatography column the detector generates a measurable signal which is usually expressed as a peak on the chromatogram. A suppressor is being used to reduce the background conductance of the Eluent and at the same time enhance the conductance of the sample ions. The chromatogram is a record of detector output (electrical conductivity) versus time as the analyte passes through the chromatography system. It may consist of a series of several peaks corresponding to the different times in which components of the analyte mixture emerge from the column. In general, the number of peaks corresponds to the minimum number of different substances (compounds or ions) contained in the analyte.

Some embodiments contemplate that any ion chromatography technique capable of detecting iodide with a high degree of sensitivity and precision can be used with the methods of the present invention. Such techniques include, but are not limited to, conductivity detection, UV/VIS detection, and amperometric detection.

In some embodiments, the step of measuring the iodide concentration in the plasma sample by ion chromatography comprises conductivity detection. Electric conductivity measurement of a solution is a method of detecting ions in the solution. After the targeted ions are eluted, the change in electric current is detected, with a constant voltage imposed between the electrodes. A conductivity detector is employed as a detector in an ion chromatograph, which is a system dedicated to measuring ions. This detector is used mainly to measure inorganic ions and small organic substances, including organic acids and amines. The conductivity detector is highly sensitive, but very susceptible to the effect of temperature variation (a change of 1° C. in solution temperature causes a change of roughly 2% in electric conductivity). Various methods of avoiding temperature variations have been devised, such as constant-temperature cells.

In some embodiments, the step measuring the iodide concentration in the plasma by ion chromatography comprises UV/VIS detection. A UV/UV-VIS detector monitors the absorption of light with a specified wavelength. However, some substances absorb light at one wavelength, and then emit light called fluorescence at another wavelength. This is a phenomenon in which a substance absorbs light to reach a high-energy level and then emits light to return to its original level. Such a substance has specific wavelengths of light that it absorbs (excitation wavelengths) and emits (emission wavelengths). As familiar examples, fluorescent paints and highlighters emit fluorescence with a clear color.

In some embodiments, the step measuring the iodide concentration in the plasma by ion chromatography comprises amperometric detection. In amperometry, current is measured during a read pulse as a constant potential (voltage) is applied across electrodes. The measured current is used to quantify the analyte, e.g. iodide, in a sample. Amperometry measures the rate at which the electrochemically active species, and thus the analyte, is being oxidized or reduced near the working electrode. Many variations of the amperometric method for biosensors have been described. For example, chromatography may be performed using a Mettosep A Supp 150/4.0 analytical column with Metrosep A Supp 4/5 Guard column. In one non-limiting example, the mobile phase is 3.8 mM sodium carbonate/1.2 mM sodium bicarbonate/5% acetonitrile; the column temperature is 40°; the sample temperature is ambient, the flow rate is 0.7 mL/min; the detector us an electrochemical detector set at 0.06V DC Mode; the cell electrode is Ag; the retention time is about 12 minutes; the injection volume us 80 μL; and/or the run time is about 20 minutes. In certain embodiments, sample analysis is performed according to standard operating procedures.

Particular embodiments contemplate that one advantage of ion chromatography is that a relatively small amount of a sample is required for analysis. Thus, in certain embodiments, an amount of protein is removed from the plasma sample and then the ion concentration is measured in the plasma sample by ion chromatography, wherein the plasma sample comprise a volume of less than about 10 ml, less than about 9 ml, less than about 8 ml, less than about 7 ml, less than about 6 ml, less than about 5 ml, less than about 4 ml, less than about 3 ml, less than about 2 ml, between about 0.01 ml and about 2 ml, or between about 0.1 ml and about 1 ml. In particular embodiments, the plasma sample comprises a volume of about 10 μl, about 50 μl, about 100 μl, about 200 μl, about 300 μl, about 400 μl, about 500 μl, about 600 μl, about 700 μl, about 800 μl, about 900 μl, about 1 ml, about 1.2 ml, about 1.4 ml, about 1.6 ml, about 1.8 ml, or about 2 ml.

Certain embodiments contemplate that one advantage of ion chromatography is that it measures iodide in a sample with a high degree of sensitivity. For example, some embodiments contemplate that ion chromatography can be used to detect a concentration of less than 0.1 part per million (ppm), less than 1 ppm, less than 5 ppm, or less than 10 ppm. In some embodiments, ion chromatography is used to detect a concentration of iodide between about 0.001 parts per billion (ppb) and about 1 ppm, between about 0.1 parts per billion (ppb) and about 500 ppb, between about 0.3 ppb and about 200 ppb, between about 1 ppb and about 200 ppb, or between about 2.5 ppb and about 200 ppb of iodide in a sample.

Particular embodiments contemplate that the concentration of iodide measured in the sample reflects the concentration of iodide in the plasma sample that has been diluted by the protein precipitation agent. Thus, one of skill in the art will appreciate that the concentration of iodide in the plasma in the subject, i.e. the concentration of the circulating iodide, can be calculated based on the known ratio of the protein precipitation agent added to the plasma sample during the protein removal step. For example, if a protein precipitation agent is added to the plasma sample at a ratio of 3:1 (agent:plasma), and an ion concentration of about 6 ppb is measured in the plasma sample by ion chromatography, then one of skill in the art will appreciate that the subject has a circulating iodide concentration of about 24 ppb.

Particular embodiments contemplate that in some instances where the subject has a high concentration of circulating iodide, for example if a plasma sample is taken from subject that had been administered an iodide compound, the plasma sample would have to be diluted prior to being measured by ion-chromatography. This is because ion chromatography systems will have particular concentration ranges for optimal accuracy to measure the iodide concentration. Particular embodiments contemplate that such a concentration range will depend, at least in part, on the specifications of the ion chromatography system in use and the conditions under which the plasma sample is measured. In particular embodiments, the plasma sample will be diluted to comprise an iodide concentration of about 2.5 ppb to about 200 ppb. In particular embodiments, the plasma sample will be diluted to comprise an iodide concentration of about 0.1 ppb to about 500 ppb or about 0.3 ppb to about 200 ppb.

Some embodiments contemplate that standards comprising known amounts of iodide are generated and used as a reference to accurately measure a concentration of iodide in a sample by ion chromatography. There are various methods of generating iodide standards that are appropriate for use with ion chromatography, and one of skill in the art will readily identify and employ methods for generating iodide standards for use with the methods of iodide detection of the present invention. In certain embodiments, iodide curves are generated using NaI, while in certain embodiments, iodide curves are generated using KI. Concentrations of NaI or KI may be determined, and the concentration of I⁻ may be readily derived from these, e.g., based on the relative weight of I⁻ and Na or I⁻ and K.

In particular embodiments, an amount of protein is removed from a 500 μl plasma sample, and a concentration of iodide between about 0.001 ppb and about 1 ppm, between about 0.1 ppb and about 500 ppb, between about 0.3 ppb and about 200 ppb, between about 2.5 ppb and about 200 ppb, or between about 1 ppb and about 200 ppb, is measured in the plasma sample with ion chromatography comprising amperometric detection.

Certain embodiments contemplate that the any ion chromatography system capable of sensitive and accurate detection of iodide is suitable for the methods of the present invention. In certain embodiments, commercially available ion chromatography systems are used to measure iodide concentration in the plasma sample. Commercial ion chromatography systems include, but are not limited to, the DIONEX™ ion chromatography systems by Fisher Scientific, the CARS™ and SAMS™ suppressor systems by EMD Bioscience, the IONQUEST™ ion chromatography system by Cecil Instruments, NGC™ chromatography system from BioRad, AKTA™ chromatography systems from GE Healthcare Life Sciences, and the Metrohm 850 Professional IC AnCat. In some embodiments, the concentration of iodide in the plasma sample is measured by ion chromatography with the Metrohm 850 Professional IC AnCat system.

Iodide Compounds

Iodine (I), the second heaviest natural halogen, is the non-metal element with atomic number 53. Under standard pressure and temperature it exists as a solid diatomic I₂ molecule. There are 34 iodine isotopes with known half-lives, said isotopes having mass numbers ranging from 108 to 144. Natural iodine, however, consists of one stable isotope, ¹²⁷I. Iodide is the reduced form of iodine.

Particular embodiments contemplate that increasing levels of circulating iodide, for example by administering compounds that contain iodide or iodine (“iodide compounds”), are useful for treating diseases or conditions that include, but are not limited to, hypoxia, ischemia, reperfusion injury, stroke, heart attack, and blood loss. Certain embodiments contemplate that increasing levels of circulating iodide in a subject who will undergo or who has undergone cell, tissue, or organ transplantation improves the outcome.

In particular embodiments, the iodide detected according to the present invention is a reduced form of iodide. In certain embodiments, the iodide detected or administered to a subject is selected from, or results from metabolism of, Aluminium iodide, Aluminium monoiodide, Ammonium iodide, Antimony triiodide, Arsenic diiodide, Arsenic triiodide, Barium iodide, Beryllium iodide, Bismuth(III) iodide, Boron triiodide, Cadmium iodide, Caesium iodide, Calcium iodide, Candocuronium iodide, Carbon tetraiodide, Cobalt(II) iodide, Coccinite, Copper(I) iodide, DiOC6, Diphosphorus tetraiodide, Dithiazanine iodide, Echothiophate, Einsteinium(III) iodide, Eschenmoser's salt, Ethylenediamine dihydroiodide, Gallium(III) iodide, GelGreen, GelRed, Germanium iodide, Gold monoiodide, Gold triiodide, Hydrogen iodide, Iodine oxide, Iodomethylzinc iodide, Iodosilane, Iron(II) iodide, Lead(II) iodide, Lithium iodide, Magnesium iodide, Manganese(II) iodide, Mercury(I) iodide, Mercury(II) iodide, Nickel(II) iodide, Nitrogen triiodide, Palladium(II) iodide, Phosphorus triiodide, Polyiodide, Potassium iodide, Potassium tetraiodomercurate(II), Propidium iodide, Rubidium iodide, Rubidium silver iodide, Samarium(II) iodide, Silicon tetraiodide, Silver iodide, Sodium iodide, Strontium iodide, Tellurium iodide, Tellurium tetraiodide, Terbium(III) iodide, Tetraethylammonium iodide, Thallium triiodide, Thallium(I) iodide, Thorium(IV) iodide, Tibezonium iodide, Tiemonium iodide, Tin(II) iodide, Tin(IV) iodide, Titanium tetraiodide, Triiodide, Trimethylsilyl iodide, Trimethylsulfoxonium iodide, Uranium pentaiodide, Uranium tetraiodide, Uranium triiodide, Vanadium(III) iodide, Zinc iodide, and Zirconium(IV) iodide.

In particular embodiments, the iodide is sodium iodide, potassium iodide, hydrogen iodide, calcium iodide, magnesium iodide, zinc iodide, lithium iodide, or silver iodide.

In some embodiments, the iodide compound is an iodate comprising one or more compounds from the non-limiting list of Calcium iodate, Iodic acid, Potassium iodate, Seeligerite, Silver iodate, and Sodium iodate.

In particular embodiments, the iodide compound is an iodate comprising sodium iodate, potassium iodate, calcium iodate, or silver iodate.

In some embodiments, iodide compound is an organoiodide comprising one or more compounds from the non-limiting list of ²⁵I-NBF, ²⁵I-NBMD, ²⁵I-NBOH, ²⁵I-NBOMe, 2C-I, 5, 5-I-R91150, Acetrizoic acid, Adipiodone, Adosterol, Altropane, AM-1241, AM-2233, AM-630, AM-679 (cannabinoid), AM-694, AM251, Amiodarone, Benziodarone, Bromoiodomethane, Budiodarone, Butyl iodide, Carbon tetraiodide, Chiniofon, Chloroiodomethane, Clioquinol, Diatrizoic acid, Diiodohydroxypropane, Diiodohydroxyquinoline, Diiodomethane, 2,5-Dimethoxy-4-iodoamphetamine, Domiodol, Erythrosine, Ethyl iodide, Ethyl iodoacetate, Fialuridine, Fluoroiodomethane, Haloprogin, Herapathite, IAEDANS, Ibacitabine, IDNNA, Idoxifene, Idoxuridine, Iniparib, Iobenguane, Iobenzamic acid, Iobitridol, Iocarmic acid, Iocetamic acid, Iodamide, Iodixanol, Iodoacetamide, Iodoacetic acid, Para-Iodoamphetamine, Iodobenzamide, Iodobenzene, 2-Iodobenzoic acid, 19-Iodocholesterol, Iodocyanopindolol, Iodoform, 1-Iodomorphine, Iodophenol, Iodophenpropit, 4-Iodopropofol, Iodopropynyl butylcarbamate, Iodotrifluoroethylene, Iodoxamic acid, 2-Iodoxybenzoic acid, Iofetamine (123I), Ioflupane (123I), Ioglicic acid, Ioglycamic acid, Iomazenil, Iomeprol, Iopamidol, Iopanoic acid, Iopentol, Iopromide, Iopydol, Iotrolan, Iotroxic acid, Ioversol, Ioxaglic acid, Ioxilan, Ipodate sodium, Isopropyl iodide, Methiodal, Methyl iodide, Metrizamide, Metrizoic acid, Pentafluoroethyl iodide, Plakohypaphorine, N-Propyl iodide, Propyliodone, Rafoxanide, Rose bengal, RTI-121, RTI-229, RTI-353, RTI-55, SB-258,585, Sodium acetrizoate, Tiratricol, Trifluoroiodomethane, and Tyropanoic acid.

In particular embodiments, the iodide compound is an organoiodide. Organoiodine compounds are organic compounds that contain one or more carbon-iodine bonds. Almost all organoiodine compounds feature iodide connected to one carbon center. These are usually classified as derivatives of I. Some organoiodine compounds feature iodine in higher oxidation states. Organoiodine compounds, often used as disinfectants or pesticides, include, e.g., iodoform (CHIS), methylene iodide (CH₂I₂), and methyl iodide (CH₃I). In particular embodiment, the organoiodide is a polyiodoorganic compound. Polyiodoorganic compounds are sometimes employed as X-ray contrast agents, in fluoroscopy, a type of medical imaging. A variety of such polyiodoorganic compounds are available commercially; many are derivatives of 1,3,5-triiodobenzene and contain about 50% by weight iodine. In certain embodiments, the agent is soluble in water, non-toxic and/or readily excreted. A representative reagent is Ioversol, which has water-solubilizing diol substituents. Other organoiodine compounds include but are not limited to the two thyroid hormones thyroxine (“T₄”) and triiodothyronine (“T₃”). Marine natural products are rich sources of organoiodine compounds, including the recently discovered plakohypaphorines from the sponge Plakortis simplex.

The present invention also includes the use of compounds, e.g., drug compounds, into which an iodine is incorporated. For example, an iodine may be incorporated into existing drugs such as N-acetyl cysteine, standard pain relievers, and non-steroidal anti-inflammatory drugs, such as, e.g., aspirin, ibuprofen and naproxen. Most NSAIDs act as nonselective inhibitors of the enzyme cyclooxygenase (COX), inhibiting both the cyclooxygenase-1 (COX-1) and cyclooxygenase-2 (COX-2) isoenzymes.

In certain embodiments, said iodide compound is a polyiodide. The polyiodides are a class of polyhalogen anions composed of entirely iodine atoms. The most common and simplest member is the triiodide ion, I₃⁻. Other known, larger polyiodides include [I₄]²⁻, [I₅]⁻, [I₇]⁻, [I₈]²⁻, [I₉]⁻, [I₁₀]²⁻, [I₁₁]⁻, [I₁₂]²⁻, [I₁₃]³⁻, [I₁₆]²⁻, [I₂₂]⁴⁻, [I₂₆]³⁻, [I₂₆]⁴⁻, [I₂₈]⁴⁻, and [I₂₉]³⁻. One example of a polyiodide is Lugol's iodine, also called Lugol's solution. Lugol's solution is commercially available in different potencies of 1%, 2%, or 5% Iodine. The 5% solution consists of 5% (wt/v) iodine (I₂) and 10% (wt/v) potassium iodide (KI) mixed in distilled water and has a total iodine content of 130 mg/ml. Potassium iodide renders the elementary iodine soluble in water through the formation of the triiodide (I⁻³) ion. Other names for Lugol's solution are I₂KI (iodine-potassium iodide); Markodine, Strong solution (Systemic); and Aqueous Iodine Solution BCP. Examples of polyiodides, including their ions and counter-cations are shown in Table 1.

TABLE 1
Polyiodides

| Anion | Counter-cation |
|---|---|
| [I₃]⁻ | Cs⁺ |
| [I₄]²⁻ | [Cu(NH₃)₄]²⁺ |
| [I₅]⁻ | [EtMe₃N]⁺ |
| | [EtMePh₂N]⁺ |
| [I₇]⁻ | [Ag(18aneS₆)]⁺ |
| [I₈]²⁻ | [Ni(phen)₃]²⁺ |
| [I₉]⁻ | [Me₂ⁱPrPhN]⁺ |
| | [Me₄N]⁺ |
| [I₁₀]²⁻ | [Cd(12-crown-4)₂]²⁺ |
| [I₁₁]³⁻ | [(16aneS₄)PdIPd(16aneS₄)]³⁺ |
| [I₁₂]²⁻ | [Ag₂(15aneS₅)₂]²⁺ |
| | [Cu(Dafone)₃]²⁺ |
| [I₁₃]³⁻ | [Me₂Ph₂N]⁺ |
| [I₁₆]²⁻ | [Me₂Ph₂N]⁺ |
| | [ⁱPrMe₂PhN]⁺ |
| [I₂₂]⁴⁻ | [MePh₃P]⁺ |
| [I₂₆]³⁻ | [Me₃S]⁺ |
| [I₂₆]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |
| [I₂₉]³⁻ | Cp₂Fe |
| [I₂₂]⁴⁻ | [MePh₃P]⁺ |
| [I₂₆]³⁻ | [Me₃S]⁺ |
| [I₂₆]⁴⁻ | DMFc⁺ |

In one embodiment, the iodide compound is a tincture of iodine solutions, which comprises or consists of elemental iodine, and iodide salts dissolved in water and alcohol.

In some embodiments, the iodide compound is a periodate comprising one or more compounds from the non-limiting list of Dess-Martin periodinane, I, 2-Iodoxybenzoic acid, Periodic acid, Potassium periodate, and Sodium periodate.

In particular embodiments, the iodide compound is a periodate comprising sodium periodate, potassium periodate, calcium periodate, or silver periodate.

In particular embodiments, the iodide compound is a periodinane. Periodinanes are chemical compounds containing hypervalent iodine. In some embodiments, said iodide compound is a periodinane comprising one or more compounds from the non-limiting list of (Bis(trifluoroacetoxy)iodo)benzene, Dess-Martin periodinane, Iodobenzene dichloride, Iodosobenzene, and 2-Iodoxybenzoic acid.

In one embodiment, the iodide compound is an oil-infused iodide or iodine oil infusion.

Certain embodiments of the present invention relate to the measurement of iodide concentration of a plasma sample taken from a subject who has been or will be administered with an iodide compound.

In some embodiments, a subject is administered or will be administered a unit dosage comprising an iodide compound. In particular embodiments, a unit dosage form comprising an iodide compound, e.g. NaI, comprises or consists of about 0.005 mg to about 5000 mg, about 0.05 to about 1000 mg, about 0.5 mg to about 100 mg, about 1 mg to about 100 mg, about 2.5 mg to about 100 mg, about 0.5 mg to about 50 mg, about 1 mg to about 50 mg, about 2.5 mg to about 50 mg, about 5 mg to about 50 mg, about 10 mg to about 50 mg, or about 1 mg, about 2 mg, about 5 mg, about 10 mg, or about 15 mg. In related embodiments, the unit dosage form comprises less than or equal to 150 mg, less than or equal to 125 mg, less than or equal to 100 mg, less than or equal to 75 mg, less than or equal to 50 mg, less than or equal to 25 mg, or less than or equal to 10 mg of the iodide compound. In certain embodiments, the unit dosage form comprises between about 1 mg and about 150 mg (including any interval in this range), between about 1 mg and about 125 mg, between about 1 mg and about 100 mg, between about 1 mg and about 75 mg, between about 1 mg and about 50 mg, between about 1 mg and about 25 mg or between about 1 mg and about 10 mg of the iodide compound. In certain embodiments, the unit dosage form comprises about 150 mg, about 125 mg, about 100 mg, about 75 mg, about 50 mg, about 25 mg or about 10 mg of the iodide compound. In certain embodiments, the unit dosage form comprises less than or equal to 1000 mg, less than or equal to 800 mg, less than or equal to 700 mg, less than or equal to 500 mg, less than or equal to 250 mg, less than or equal to 200 mg, or less than or equal to 150 mg of the iodide compound. In certain embodiments, the unit dosage form comprises between about 50 mg and 500 mg, about 50 mg and 100 mg, about 100 mg and about 1000 mg (including any interval in this range), between about 150 mg and about 800 mg, between about 200 mg and about 700 mg, between about 250 mg and about 600 mg, between about 300 mg and about 500 mg, between about 350 mg and about 450 mg or between about 300 mg and about 700 mg of the iodide compound. In certain embodiments, the unit dosage form comprises about 200 mg, about 300 mg, about 400 mg, about 500 mg, about 600 mg, about 700 mg, about 800 mg, about 900 mg, or about 1000 mg of the iodide compound.

In some embodiments, including, e.g., embodiments where the unit dosage form is formulated as a liquid, e.g., for intravenous administration or administration by infusion, the concentration of iodide compound or salt or precursor thereof present in a unit dosage form of the present invention is about 0.0001 mM to about 100 M, about 0.0005 mM to about 50 M, about 0.001 mM to about 10 M, about 0.001 mM to about 5 M, about 0.001 mM to about 1 M, about 0.005 mM to about 10 M, about 0.005 mM to about 5 M, about 0.005 mM to about 1 M about 0.005 mM to about 0.5 M, about 0.01 mM to about 10 M, about 0.01 mM to about 5 M, about 0.01 mM to about 2 M, about 0.1 mM to about 1 M, about 0.1 mM to about 0.5 M, about 0.5 mM to about 5 M, about 0.5 mM to about 2 M, about 0.5 mM to about 1 M, about 0.5 mM to about 0.5 M, about 1 mM to about 5 M, about 1 mM to about 2 M, about 1 mM to about 1 M, about 1 mM to about 0.5 M, about 5 mM to about 5 M, about 5 mM to about 2 M about 5 mM to about 1 M, about 5 mM to about 0.5 M, about 5 mM to about 0.25 M, about 10 mM to about 1 M, about 10 mM to about 0.5 M, about 10 mM to about 0.25 M, or about 10 mM, about 50 mM about 100 mM, or about 200 mM. The unit dosage form may further comprise one or more pharmaceutically acceptable diluents, excipients or carriers.

In certain embodiment, the unit dosage form comprises iodide, e.g., NaI, and the effective amount is greater than or equal to about 150 μg, greater than or equal to about 300 μg, greater than or equal to about 500 μg, greater than or equal to about 1 mg, greater than or equal to about 2 mg, greater than or equal to about 5 mg, greater than or equal to about 10 mg, greater than or equal to about 15 mg, or greater than or equal to about 20 mg. In certain embodiments, the effective amount is 150 μg to 1000 mg, 300 μg to 1000 mg, 500 μg to 1000 mg, 1 mg to 1000 mg, 2 mg to 1000 mg, 5 mg to 1000 mg, 10 mg to 1000 mg, 150 μg to 100 mg, 300 μg to 100 mg, 500 μg to 100 mg, 1 mg to 100 mg, 2 mg to 100 mg, 5 mg to 100 mg, or 10 mg to 100 mg.

In certain embodiments, the effective amount is between about 0.1 mg/kg to about 100 mg/kg, about 0.1 mg/kg to about 10 mg/kg, about 0.5 mg/kg to about 5 mg/kg, about 0.2 mg/kg, about 0.5 mg/kg, about 1.0 mg/kg, about 2.0 mg/kg, about 3.0 mg/kg, about 4.0 mg/kg, about 5.0 mg/kg, about 6.0 mg/kg, about 7.0 mg/kg, about 8.0 mg/kg, about 9.0 mg/kg or about 10 mg/kg. In certain embodiments, the effective amount is 150 μg to 50 mg, 300 μg to 20 mg, 500 μg to 10 mg, 1 mg to 20 mg, 1 mg to 10 mg, or about 5 mg, about 10 mg, about 15 mg, or about 20 mg. In other embodiments, the effective amount is between about 1 mg and about 150 mg (including any interval in this range), between about 1 mg and about 125 mg, between about 1 mg and about 100 mg, between about 1 mg and about 75 mg, between about 1 mg and about 50 mg, between about 1 mg and about 25 mg or between about 1 mg and about 10 mg of the iodide compound. In certain embodiments, the effective amount is about 150 mg, about 125 mg, about 100 mg, about 75 mg, about 50 mg, about 25 mg or about 10 mg of the iodide compound. In certain embodiments, the effective amount comprises less than or equal to 1000 mg, less than or equal to 800 mg, less than or equal to 700 mg, less than or equal to 500 mg, less than or equal to 250 mg, less than or equal to 200 mg, or less than or equal to 150 mg of the iodide compound. In certain embodiments, the effective amount is between about 100 mg and about 1000 mg (including any interval in this range), between about 150 mg and about 800 mg, between about 200 mg and about 700 mg, between about 250 mg and about 600 mg, between about 300 mg and about 500 mg, between about 350 mg and about 450 mg or between about 300 mg and about 700 mg of the iodide compound. In certain embodiments, the effective amount is about 200 mg, about 300 mg, about 400 mg, about 500 mg, about 600 mg, about 700 mg, about 800 mg, about 900 mg, or about 1000 mg of the iodide compound. In particular embodiments, the effective amount is the amount per day.

In general the amount of the active compound present in a composition or unit dosage form depends inter alia on the specific compound and formulation, the age and condition of the subject, and the specific features of the injury, condition, or disease being treated or prevented, the route of administration and the dosage frequency.

The dosage frequency also depends on the injury, condition or disease being treated or prevented, the amount or concentration of the compound, the specific composition used, the route of administration and may incorporate subject-specific variation including, but not limited to age, weight, gender, or overall health.

In certain embodiments, a unit dosage form suitable for oral administration is in the form of a pill, drenches (aqueous or non-aqueous solutions or suspensions), boluses, powders, granules, polymer release formulations, pastes for application to the tongue tablet, caplet or a capsule. A pill is a small, round, solid pharmaceutical oral dosage form that was in use before the advent of tablets and capsules. In colloquial usage, tablets, capsules, and caplets are still often referred to as “pills” collectively. In certain embodiments, pills are made by mixing the active ingredients with an excipient such as glucose syrup in a mortar and pestle to form a paste, then divided into suitable sizes, and often coated with sugar to make them more palatable.

Dosage levels of an iodide compound present in a composition described herein may be varied as so to obtain an amount of the iodide compound that is effective to achieved the desired therapeutic effect for a particular subject, iodide compound, and mode of administration, without being toxic to the subject.

The present invention further includes stable liquid pharmaceutical compositions formulated for parenteral administration, e.g., intravenous administration or administration by infusion. In certain embodiments, the stable liquid pharmaceutical compositions comprise an iodide. In some embodiments, the stable liquid pharmaceutical compositions comprise an iodide, and glutathione. In particular embodiments, the composition is contained within an oxygen-impermeable container, and may be under nitrogen or argon gas. In particular embodiments, the amount of composition present in the container is a unit dosage amount comprising or consisting of a suitable dosage amount for administration to a subject in need thereof.

Formulations of iodide compounds for parenteral administration may comprise an iodide compound in combination with one or more pharmaceutically acceptable isotonic aqueous or nonaqueous solutions, dispersions, suspensions or emulsions, or powders which may be reconstituted into sterile injectable solutions or dispersions just prior to use. Parenteral formulations may contain antioxidants; buffers or solutes which render the formulation isotonic with the blood of the intended subject; bacteriostats; suspending; or thickening agents.

Measurements of Iodide Concentration in Subjects.

In particular embodiments, a plasma sample is taken from a subject, and the iodide concentration of the plasma is measured by any of the methods described herein. In particular embodiments the subject is a human. In some embodiments, plasma sample is taken from a subject following administration of iodide to the subject. In some embodiments, the plasma sample is taken from a subject who will be administered iodide. In particular embodiments, the plasma sample is taken from a subject that will potentially benefit from a therapeutic administration of iodide. In some embodiments, the plasma sample is taken from a subject who will be or who has been administered iodide for the purposes of treating a disease or condition. In certain embodiments, the plasma sample is taken from a subject who is diagnosed with or is determined to be at risk for hypoxia, ischemia or reperfusion injury. In particular embodiments, the plasma sample is taken from a subject who is diagnosed with or is determined to be at risk for stroke, heart attack, or blood loss. In certain embodiments, the subject is undergoing or is scheduled to undergo cell, tissue, or organ transplantation.

In some embodiments, the plasma sample is obtained from a non-human animal, such as a laboratory animal. In particular embodiments, the plasma sample is obtained from a mouse, a rat, a guinea pig, a vole, a rabbit, a hamster, a cat, a dogs, a reptile, a bird, or a non-human primate. In some embodiments, the non-human animal has been administered iodide.

Particular embodiments are directed to methods for determining a concentration of iodide in the blood plasma of a subject over a period of time by collecting a plasma sample from the subject at two or more time points within the period of time, and measuring the concentration of the iodide in each plasma sample by the methods described herein. In particular embodiments, a plasma sample is taken from the subject at two, three, four, five, six, seven, eight, nine, ten, more than ten, more than fifteen, more than twenty, more than twenty-five, more than thirty, more than thirty-five, more than forty, more than forty-five, or more than fifty time points during the period of time. In some embodiments, a plasma sample is taken every 5 minutes, every 10 minutes, every 15 minutes, every 30 minutes, every hour, every two hours, every six hours, every twelve, hours, daily, weekly, or monthly during the period of time. In particular embodiments, the period of time lasts for about 1 hour, about 2 hours, about 3 hours, about 4 hours, about 5 hours, about 6 hours, about 7 hours, about 8 hours, about 12 hours, about 24 hours, about 2 days, about 3 days, about 4 days, about 5 days, about 6 days, about 7 days, about 2 weeks, about 3 weeks, about 4 weeks, about 5 weeks, about 6 weeks, about 2 months, about 3 months, about 4 months, about 5 months, or about 6 months. In certain embodiments, the period of time lasts between about 1 hour and about 24 hours, about 1 day and about 1 week, between about 1 week and 1 month, or between about 1 month and about 6 months.

Certain embodiments are directed to methods for determining a concentration of iodide in the blood plasma of a subject over a period of time by collecting a plasma sample from the subject at different time points within the period of time. In particular embodiments, the subject is administered iodide prior to the period of time. In some embodiments, the period of time begins with the subject is administered iodide. In certain embodiments, the subject is administered iodide regularly during the period of time. In some embodiments, the subject is administered iodide continuously during the period of time.

In some embodiments, the period of time begins when the subject is administered iodide, and plasma samples are taken during the period of time to determine the time course of plasma iodide levels resulting from the administered iodide. For example, the subject may be a laboratory animal that is administered a composition comprising iodide, and plasma samples are collected from the laboratory animal at regular intervals during the period of time. Iodide concentrations would be measured in the plasma samples by the methods of the present invention described herein. Particular embodiments contemplate that one of skill in the art would be able to ascertain pharmacokinetic information from such an experiment, for example but not limited to, the peak concentration of plasma iodide resulting from the administered dose, the time to reach the peak plasma concentration following the iodide administration, and the duration of time the elevated iodide plasma concentrations persist.

In particular embodiments, plasma samples are taken from a subject during a period of time where the subject is regularly administered iodide. For example, the subject may be a human subject undergoing treatment for a disease or condition with iodide, and plasma samples may be taken to determine if the patient is receiving too much or too little dosage.

Particular embodiments contemplate that iodide is administered to a subject orally, nasally, intravenously, intramuscularly, ocularly, transdermally, or subcutaneously. In some embodiments, the iodide is administered intravenously. In particular embodiments, the iodide is administered orally.

Some embodiments are directed to a method for determining if the amount of iodide administered to a subject should be increased or decreased comprising the steps of: administering the amount of iodide to the subject; collecting a plasma sample from the subject, measuring the concentration of the iodide in the plasma sample by performing a method described herein to measure the concentration of iodide in the plasma sample, thereby obtaining a measurement of the concentration; and comparing the measurement of the concentration to a predetermined preferred range of plasma iodide concentrations. If the measurement of the iodide concentration falls within the predetermined preferred range, then the amount of iodide administered to the subject will not be altered. However, if the measurement of the iodide concentration falls below the predetermined preferred range, then the amount of iodide will be increased, and if the measurement of the concentration falls above the predetermined preferred range, then the amount of iodide will be decreased. In some embodiments, the plasma sample is taken from the subject about 5 minutes, about 10 minutes, about 15 minutes, about 30 minutes, about 45 minutes, about 1 hour, or about 2 hours after the iodide is administered to the subject. Some embodiments contemplate that the preferred concentration range of iodide in plasma is about 5 ppb to about 1000 ppb, about 10 ppb to about 500 ppb, about 25 ppb to about 200 ppb, or about 50 ppb to about 100 ppb. Particular embodiments contemplate that the plasma sample is taken from the subject 30 minutes after the iodide is administered and that the predetermined preferred range is 50-100 ppb of iodide.

### EXAMPLES

### Example 1

### Amperometric Detection Set Up

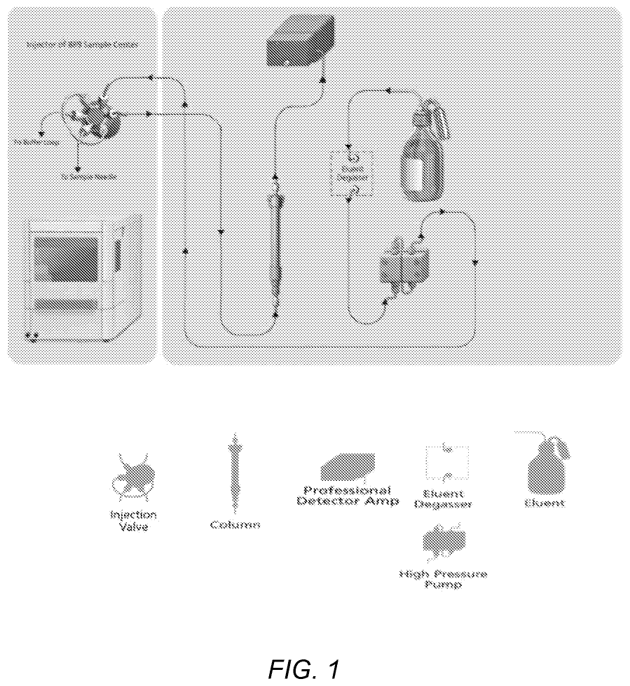

Amperometric measurements were performed using a Metrohm 850 Professional IC AnCat—MSM High Capacity—MCS (2.850.3040) with 889 IC Sample Center (2.889.0010) with 20 uL injection volume, the general flow path of which is shown in FIG. 1. Degassed eluent flows through the pump, into the pulsation dampener, and to the injection valve where sample is introduced. The autosampler needle pulls the sample from the vial and uses the peristaltic pump to send the sample to the injection valve. The injection valve fills the 20 μl sample loop with the sample, where the sample is mixed with eluent and sent to the column. In the column, separation occurs and the sample/analyte ions exchange with eluent ions. Sample ions are then displaced with the flow of eluent through the column and continue to flow into the Amperometric detector for ion measurement by working electrode.

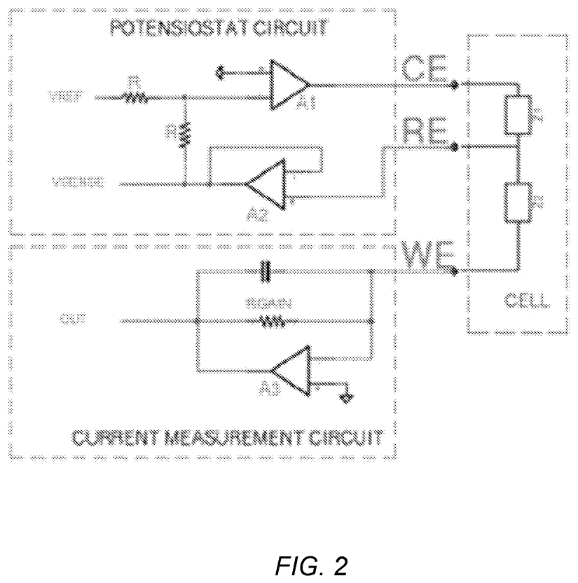

For the described experiments, the column used was a Metrosep A Supp 5—150/4.0 (#61006520) with Metrosep A Supp 4/5 Guard/4.0 (#61006500). IC vials were AQ Brand 2 mL glass vials (#9509C-WCV-AQ) or 2 mL glass vials with 300 μl inserts (#9532C-OCV) and the caps were Basik caps (#9502C-40T-S). The amperometric detector was a DC mode 0.1V potential, 850 IC Amperometric Detector Compact (#28509110), with an Ag Working Electrode 3 mm (#61257240), an Ag/AgCl/KCl Reference Electrode (#61257720), and a wall jet cell (#65337000). This three electrode configuration was used for the measurement of a current at a constant electric potential. The reference electrode (RE) was used for potential control, the working electrode (WE) was for current measurement, and the auxiliary electrode (AE) was for current drain. The general electrical path is shown in FIG. 2. The sample and eluent flow into the Amperometric detector, which is continually flowing current, and chemically converts the analyte at the working electrode where the working potential is measured. Pulsed amperometric detection applies high positive potentials to remove reaction products from the electrode surface and reduce the surface so it's pure, renewed, and ready for a new measurement.

Solutions used include A Supp 5 eluent 100×w/5% Acetonitrile (# ERA-IC1100) 3.8 mM Sodium Carbonate/1.2 mM Sodium Bicarbonate/5% Acetonitrile. The acetonitrile used was from Sigma (#34998-1L) and solutions were diluted in 18.2 mΩ Ultra Pure Water (dH₂0). All results were analyzed by MagIC Net 3.1 software.

### Example 2

### Generation of Iodide Standard Curves by Amperometric Detection

Iodide standard curves were generated using various forms of iodide. This Example provides illustrative methods of producing iodide standard curves using NaI and KI.

Sodium Iodide Standard Curves

First, iodide standards were prepared according to the following protocol:

1) 2-5 mg of Sodium Iodide (NaI) was weighed out.

2) A necessary volume of 3:1 Acetonitrile/dH₂O was added to make a 1,000 ppm (1,000 μg/mL) stock solution (2-5 mL accordingly)

3) Stock concentration was diluted at a 1:100 dilution to make 10 ppm by adding 20 μL of standard stock to 2 mL 3:1 Acetonitrile/dH₂O in a 2 mL Eppendorf Tube.

4) The 10 ppm stock was diluted 1:10 by adding 100 μL to 900 μL of 3:1 Acetonitrile/dH₂O to make a 1 ppm stock in a 2 mL Eppendorf Tube.

5) Standards were made up by diluting stock (1 ppm) 1:5 by adding 200 μL of stock into 800 μL of 3:1 Acetonitrile/dH₂O (diluent) in a 2 mL Eppendorf tube to make 0.2 ppm (200 ppb).

6) 1 ppm stock was diluted 1:6.6 by adding 300 μL of stock to 1700 μL diluent in a 2 mL Eppendorf tube to make 0.15 ppm (150 ppb)

7) The 150 ppb solution was serially diluted with a 1:1 dilution by taking 1000 μL of 150 ppb into 1000 μL diluent for 0.075 ppm (75 ppb) and 500 μL of 75 ppb into 500 μL diluent for 0.037 ppm (37 ppb).

8) 1 ppm stock was diluted 1:100 by taking 20 μL of stock into 1980 μL diluent to get 0.01 ppm (10 ppb) followed by a 1:1 dilution of 10 ppb by 500 μL of 10 ppb into 500 μL diluent to get 0.005 ppm (5 ppb), and then diluted 5 ppb 1:1 to get 0.0025 ppm (2.5 ppb), and the 2.5 ppb solution was then diluted 1:5 to get 0.0005 ppm (0.5 ppb), which was then diluted to get 0.0003 ppm (0.3 ppb).

9) Standard concentrations are analyzed by IC from lowest to highest: 0.0025, 0.005, 0.01, 0.03, 0.07, 0.15, 0.2 ppm (2.5 ppb, 5 ppb, 10 ppb, 37 ppb, 75 ppb, 150 ppb, and 200 ppb) with a diluent sample before and after each set of standards or sample groups.

10) Three check standards were made at 50 ppb for quality control by adding 250 μL of 200 ppb stock standard into 750 μL of diluent into a 2 mL IC glass vial. Each Check Standard was made up separately.

Iodide Eluent was generated according to the following protocol:

1) 1.876 L of dH₂O was poured into a 2 L bottle.

2) 24 mL of 100× A Supp 5 eluent was added to the dH₂O in the 2 L bottle.

3) 100 mL of Acetonitrile was added to the dH₂O in the 2 L Bottle and eluent solution was mixed.

4) Eluent was sparged with N₂ gas for 20-30 min to deoxygenate.

5) Sample calculations for eluent generation are shown in Table E1.

TABLE E1
Example calculations for eluent solution

| 100x A Supp 5 Eluent Dilution | Acetonitrile Addition |
|---|---|
| 100x Eluent/Y dilution = 1.2x Eluent | 5% ACN → 5 mL ACN/100 mL = |
| | X/2000 mL |
| 1:83.3 dil → 1/83.3 = X/2000 → | X = 100 mL ACN/2 L |
| 24 mL 100x Eluent/2 L | |

The standard iodide diluent is 3:1 Acetonitrile (ACN) to dH₂O, because plasma samples were diluted 3:1 with Acetonitrile for protein precipitation before analysis by Amperometric detection.

The ion chromatography (IC) method was run according to the following parameters:
IC Run time was 20 minutes total with each peak coming off at approximately 14 minutes.
The temperature set to 40° C.
The pressure was set to 6.0-10.0 MPa.
The electrical current was set to 6-8 nA.
The flow rate was 0.7 mL/min.

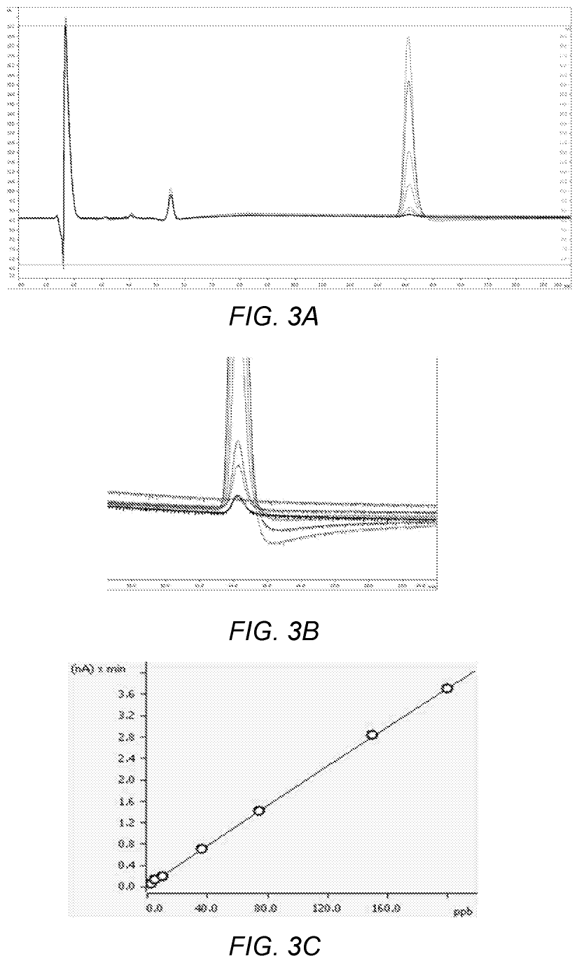

A standard curve from 2.5 ppb to 200 ppb was generated and is shown in FIGS. 3A-FIG. 3C. A weighting of 1 was used to calculate the standard curves. The trace curves in FIG. 3A represent no iodide control, 2.5 ppb, 5 ppb, 10 ppb, 37 ppb, 75 ppb, 150 ppb, and 200 ppb of iodide, from the bottom curve to the top curve, respectively. FIG. 3B provides a closer view of the no iodide control, 2.5 ppb, 5 ppb, and 10 ppb iodide curves from FIG. 3A, from the bottom curve top to the top curve, respectively. FIG. 3C shows the plot of concentration (x-axis) by amplitude (y-axis).

Potassium Iodide Standard Curves

Standard curves were generated using KI as follows:

1) Start with the 1000 mg/L (a.k.a. 1000 ppm, or μg/mL) stock of Iodide Standard for IC (41271 Sigma-Aldrich Iodide Standard for IC TraceCERT®, 1000 mg/L high purity KI in water. Dilute as described below using 3:1 Acetonitrile:Water.

2) Dilute 1:20 (50 μL into 950 μL) to 50 ppm.

3) Dilute 1:50 (20 μL into 980 μL to 1 ppm (a.k.a. 1000 ppb, or ng/mL).

4) Dilute the 1000 ppb solution to 300 ppb (300 μL into 700 μL).

5) Dilute the 300 ppb solution to 150 ppb (500 μL into 500 μL).

6) Dilute the 150 ppb solution to 75 ppb (500 μL into 500 μL).

7) Serially dilute the 300 ppb solution to 30, 3, 0.3 ppb (100 μL into 900 μL)

8) Serially dilute the 150 ppb solution to 15, 1.5, ppb (100 μL into 900 μL).

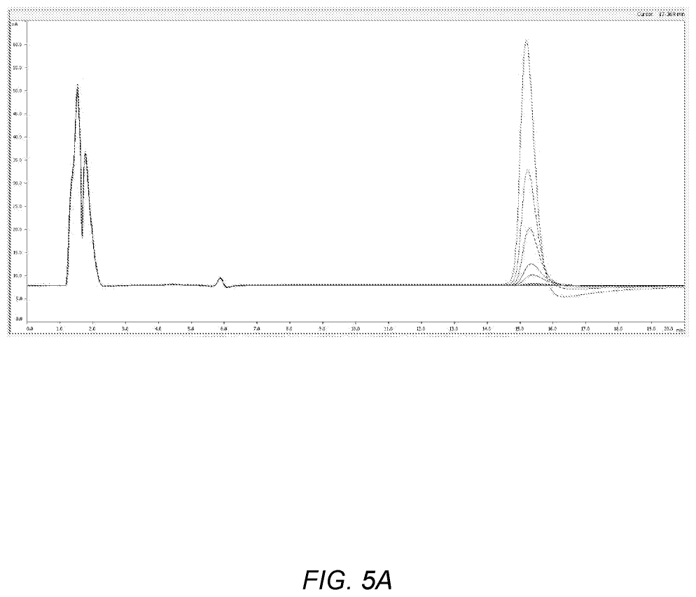

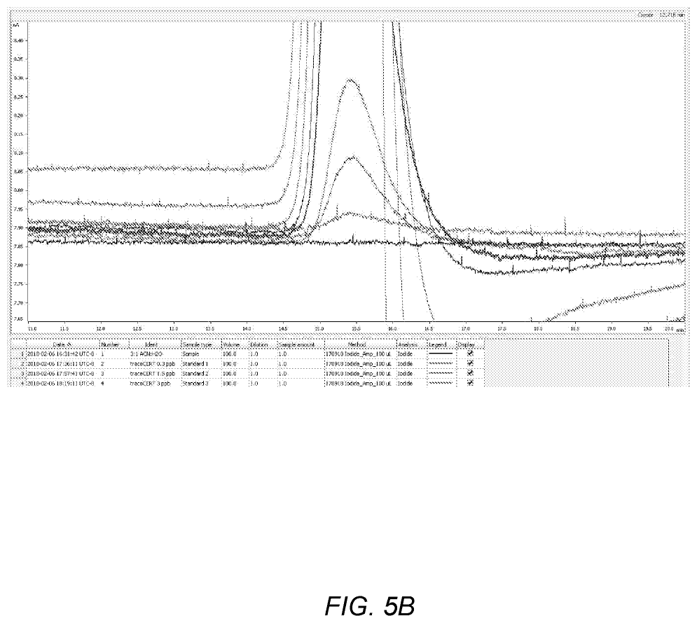

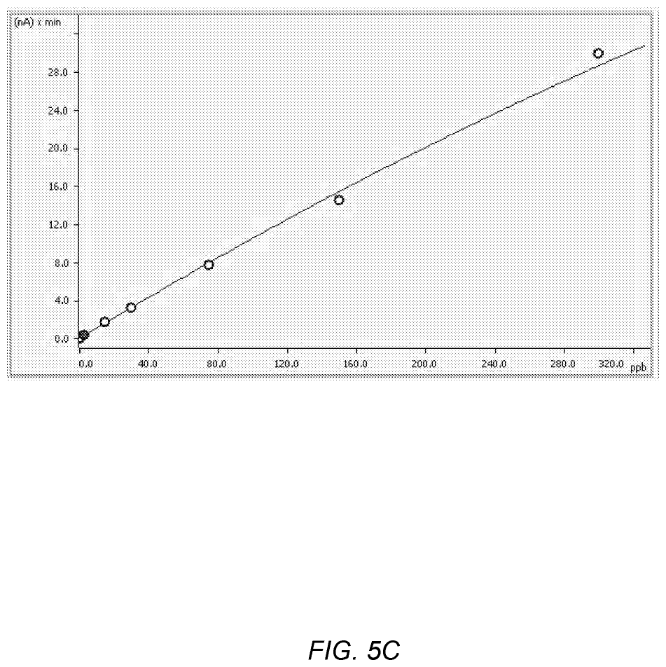

Standard concentrations were run on IC from lowest to highest: 0.3, 1.5, 3, 15, 30, 75, 150, and 300 ppb. Three check standards were made at 50 ppb for QC by adding 200 μL of the 300 ppb stock into 1 mL of 3:1 acetonitrile:water. A weighting of 1/concentration was used to calculate the standard curves. A standard curve from 0.3 ppb to 300 ppb was generated and is shown in FIG. 5A-FIG. 5C. These curves demonstrate a positive correlation between sodium iodide concentration and sodium iodide levels detected, with peaks reaching about 60 nA. The trace curves in FIG. 5A represent no iodide control, 0.3, 1.5, 3, 15, 30, 75, 150, and 300 ppb of iodide, from the bottom curve to the top curve, respectively. FIG. 5B provides a closer view of the no iodide control, 0.3 ppb, 1.5 ppb, and 3 ppb iodide curves from FIG. 5A, from the bottom curve top to the top curve, with values of about 7.8 nA, 7.95 nA, 8.1 nA and 8.3 nA, respectively. FIG. 5C shows the plot of concentration (x-axis) by amplitude (y-axis).

### Example 3

### Analysis of Sodium Iodide in Plasma by Amperometric Detection

Plasma samples were obtained from Bioreclamation IVT in 10 mL aliquots of frozen plasma in lithium heparin. The plasma samples were normal plasma samples obtained from healthy male and female volunteers who were not treated with iodide. The plasma samples were prepared according to the following protocol.
0.5 mL plasma was diluted 3:1 with pure acetonitrile for protein precipitation before analysis by Amperometric detection. (ex: 0.5 mL rat plasma plus 1.5 mL pure Acetonitrile)
Samples were mixed gently and then centrifuged at max speed (14,000 rpm or 20,817 rcf) for 2 minutes.
Supernatant was carefully removed as to not disturb protein pellet, and transferred to a clean Eppendorf tube.
Supernatants were again centrifuged at max speed for 2 minutes.
Samples were spiked as necessary with NaI to fall between 2.5 and 200 ppb NaI concentrations.
270 μL or 1 mL volumes of samples were analyzed by Amperometric detection on IC against a standard curve in a diluent matrix.

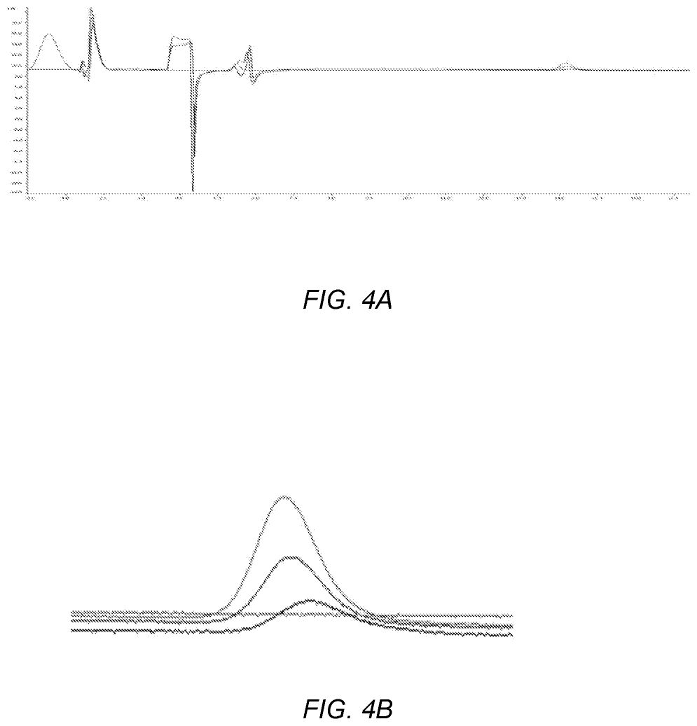

The results of iodide detection in human plasma are shown in FIG. 4A-FIG. 4B. The curves in FIG. 4B represent the diluent blank, baseline plasma, 50 ppb plasma, and 100 ppb plasma, from the bottom curve to the top curve, respectively. After 4 fold dilution due to protein precipitation, the concentration peak was at ˜2.8 ppb for baseline, and 15 ppb and 28.5 ppb for spiked plasma samples (i.e., plasma samples with known amounts of sodium iodide added). Baseline iodide levels were measured from plasma samples that were not diluted with standard iodide diluent. Aliquots from the baseline plasma samples were used to make the 100 ppb sodium iodide plasma samples and 50 ppb iodide concentrated plasma samples. These standards were made by dissolving 1 mg sodium iodide into 1 ml plasma to make a 1,000 ppm plasma sample. 104 of the 1,000 ppm plasma sample was diluted into another 1 ml plasma sample to make a 10 ppm sample. 30 μl of the 10 ppm “spiked” plasma was spiked, or diluted, into 3 mL of plasma to make 100 ppb plasma sample. Therefore, after protein precipitation, the 100 ppb plasma sample would be expected to contain 25 ppb iodide above baseline iodide levels. A 1 ml aliquot of the 0.1 ppm plasma was then spiked, i.e., diluted, into 1 mL of plasma to make 0.05 ppm or 50 ppb spiked plasma samples. The “ppm” and “ppb” designations of these samples did not account for baseline iodide levels that were in the plasma prior to the addition of the iodide diluents.

### Example 4

### Analysis of Sodium Iodide in Plasma by Amperometric Detection

Plasma samples were obtained from human volunteers treated with various dosages of sodium iodide. The plasma samples were processed and sodium iodide concentrations determined essentially as described in Example 3. The determined serum concentrations of sodium iodide positively correlated with dosages administered, further demonstrating that ion chromatography methods may be used to measure serum concentration of iodide in subjects treated with sodium iodide.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

The present invention may be embodied in other specific forms without departing from its structures, methods, or other essential characteristics as broadly described herein and claimed hereinafter. The described embodiments are to be considered in all respects only as illustrative, and not restrictive. The scope of the invention is, therefore, indicated by the appended claims, rather than by the foregoing description. All changes that come within the meaning and range of equivalency of the claims are to be embraced within their scope.

We claim:

1. A method for measuring a concentration of iodide in a plasma sample obtained from a subject, comprising the steps of:
(a) removing an amount of protein from the plasma sample; and
(b) measuring the concentration of the iodide in the sample with ion chromatography.

2. The method of claim 1, wherein the subject is a mammal.

3. The method of claim 1 or 2, wherein the plasma sample is obtained from the subject following administration of iodide to the subject.

4. The method of claim 1 or 2, wherein the plasma sample is obtained from the subject prior to the administration of iodide to the subject.

5. The method of any of claims 1-4, wherein the subject is diagnosed with or at risk of hypoxia, ischemia or reperfusion injury.

6. The method of any of claims 1-4, wherein the subject is diagnosed with or at risk of stroke, heart attack, or blood loss.

7. The method of any of claims 1-4, wherein the subject is undergoing or is scheduled to undergo cell, tissue, or organ transplantation.

8. The method of any of claims 1-7, wherein the ion chromatography comprises conductivity detection.

9. The method of any of claims 1-7, wherein the ion chromatography comprises UV/VIS detection.

10. The method of any of claims 1-9, wherein the ion chromatography comprises amperometry.

11. The method of any of claims 1-10, wherein the ion chromatography can detect a concentration of the iodide below 5 parts per million in a plasma sample.

12. The method of any of claims 1-10, wherein the ion chromatography can detect a concentration of the iodide that is between about 0.001 and about 500 parts per billion.

13. The method of any of claims 1-10, wherein the ion chromatography can detect a concentration of the iodide that is between about 2.5 and about 200 parts per billion.

14. The method of any of claims 1-13, wherein the plasma sample comprises a volume that is between about 0.05 ml and about 2 ml.

15. The method of any of 14, wherein the plasma sample comprises a volume that is between 0.1 ml and 1 ml.

16. The method of claim 15, wherein the volume is about 0.5 ml.

17. The method of any of claims 1-16, wherein step (a) is performed by precipitating the amount of protein from the sample.

18. The method of claim 17, wherein precipitating the amount of protein from the sample comprises protein precipitation by an organic solvent, protein precipitation by a metal ion, isoelectric point precipitation, protein precipitation with miscible solvents, precipitation with non-ionic hydrophilic polymers, or flocculation by polyelectrolytes.

19. The method of claim 18, wherein precipitating the amount of protein from the sample comprises the technique of protein precipitation by organic solvent.

20. The method of any of claims 17-19, wherein step (a) comprises the steps of
(i) contacting the plasma sample with a protein precipitation agent;
(ii) centrifuging the plasma sample in a manner sufficient to generate a pellet and a supernatant in the plasma sample, wherein the pellet comprises the amount of protein, and wherein the supernatant comprises the remainder of the plasma sample; and
(iii) collecting the supernatant and discarding the pellet; thereby removing the amount of protein from the plasma sample.

21. The method of claim 20, wherein the plasma sample is mixed with the protein precipitation agent prior to step (ii).

22. The method of claim 20 or 21, wherein the protein precipitation agent comprises pure acetonitrile, acetone, trichloroacetic acid (TCA), or phenol/chloroform.

23. The method of claim 22, wherein the protein precipitation agent comprises pure acetonitrile.

24. The method of any of claims 1-23, wherein the amount of protein is at least 50% of protein present in the sample prior to step (a).

25. The method of any of claims 1-23, wherein the amount of protein is at least 80% of protein present in the sample prior to step (a).

26. The method of any of claims 1-23, wherein the amount of protein is at least 90% of protein present in the sample prior to step (a).

27. The method of claim 1, wherein, step (a) comprises the steps of:
(i) contacting the plasma sample with pure acetonitrile and mixing the pure acetonitrile with the plasma sample;
(ii) centrifuging the plasma sample in a manner sufficient to generate a pellet and a supernatant in the plasma sample, wherein the pellet comprises the amount of protein, and wherein the supernatant comprises the remainder of the plasma sample; and
(iii) collecting the supernatant and discarding the pellet;
thereby removing an amount of protein from the plasma sample; and
wherein step (b) comprises measuring the amount of iodide in the supernatant using an amperometric detector; and
wherein the volume of the plasma sample is about 0.5 ml.

28. A method for determining a concentration of iodide in the blood plasma of a subject at two or more time points over a period of time comprising the steps of:
(I) administering an amount of iodide to the subject;
(II) collecting two or more plasma samples from the subject at the two or more time points within the period of time;
(III) measuring the concentration of the iodide in the two or more plasma samples by performing a method of any of claims 1-27 on each plasma sample of the two or more plasma samples.

29. The method of claim 28, wherein the period of time is between about 1 minute and about two weeks.

30. The method of claim 28 or 29, wherein the subject is continuously administered iodide intravenously over the period of time.

31. The method of claim 28 or 29, wherein the subject is administered iodide prior to the period of time.

32. The method of claim 28 or 29, wherein the period of time starts from the time the iodide is administered to the subject.

33. The method of any of claim 28-29 or 31-32, wherein the iodide is administered to the subject orally, nasally, intravenously, intramuscularly, ocularly, transdermally, or subcutaneously.

34. The method of claim 33, wherein the iodide is administered intravenously.

35. The method of claim 33, wherein the iodide is administered orally.

36. A method for determining if an amount of iodide administered to a subject should be increased or decreased by measuring a concentration of iodide in the blood plasma of a subject over a period of time comprising the steps of:
(I) administering the amount of iodide to the subject;
(II) collecting a plasma sample from the subject;
(III) measuring the concentration of the iodide in the plasma sample by performing a method of any of claims 1-27 on the plasma sample, thereby obtaining a measurement of the concentration; and
(IV) comparing the measurement of the concentration to a predetermined preferred range of plasma iodide concentrations;
wherein if the measurement of the concentration falls within the predetermined preferred range, then the amount should not be increased or decreased; wherein if the measurement of the concentration falls below the predetermined preferred range, then the amount of iodide should be increased, and wherein if the measurement of the concentration falls above the predetermined preferred range, then the amount of iodide should be decreased.

37. The method of claim 36, wherein the plasma sample is taken from the subject 30 minutes after the iodide is administered and wherein the predetermined preferred range is 50-100 ppb of iodide.
